(19) United States
(12) Patent Application Publication
(10) Pub. No.: US 2018/0228665 A1
(43) Pub. Date: Aug. 16, 2018

(54) Undergarment with opening at crotch area and an attachable, replaceable crotch piece referred to as stripe covering crotch area for maintaining vaginal hygiene and preventive care against vaginal infections

(71) Applicants: Kshitija Ganesh Relekar, Troy, MI (US); Ganesh Relekar, Troy, MI (US)

(72) Inventors: Kshitija Ganesh Relekar, Troy, MI (US); Ganesh Relekar, Troy, MI (US)

(21) Appl. No.: 15/895,180

(22) Filed: Feb. 13, 2018

Related U.S. Application Data
(60) Provisional application No. 62/458,004, filed on Feb. 13, 2017.

(51) Int. Cl. A61F 13/505; A41B 9/04; A41B 9/00; B32B 7/12; B32B 7/08; B32B 5/26; A61F 13/472; A61F 13/56

(52) U.S. Cl. CPC A61F 13/505; A61F 2013/4708; A41B 9/007; A41B 9/005; B32B 7/12; B32B 7/08; B32B 5/26; A61F 13/472; A61F 13/5605; A61F 13/5611; A41B 2300/32; A41B 2300/322; A41B 2300/324; A41B 2300/326; A41B 2400/62; B32B 2555/00; B32B 2437/00; A41B 9/04

(57) ABSTRACT

Undergarment with opening at crotch area and an attachable, replaceable crotch piece covering crotch area for maintaining vaginal hygiene. This undergarment comprises of a) Undergarment with open crotch area b) changeable crotch piece to cover open crotch area of undergarment. The crotch piece can be attached to the undergarment with adhesives or fasteners either from outside or inside. Crotch piece can be changed as needed

### FIELD OF INVENTION

Present Invention relates to women's undergarment with open crotch area and convenience of changing the crotch piece of the panty/undergarment without changing/replacing the whole undergarment

### BACKGROUND OF INVENTION

The female anatomy contributes to women's increased likelihood of contracting vaginal infections. Vaginal infections develop due to overgrowth of microbes and bacteria in vaginal area. Unclean and moist undergarment can contribute to such bacterial overgrowth.

Medical practitioners recommend keeping the vaginal opening area as clean & dry as possible as a preventive measure to protect from any vaginal infection. Thus, clothing becomes one of the important preventive care factors for feminine health. A clean undergarment helps avoid maceration of skin & bacterial overgrowth.

Frequently changing undergarment entirely for purpose of keeping vaginal area dry & clean is inconvenient and impractical on several occasions such as work, travel, outdoors & any other active lifestyle.

### What is the Invention?

It is objective of the invention to allow easy and convenient replacement of the undergarment surface in contact with vaginal area (referred to as Crotch Piece or Stripe). This will be helpful to maintain vaginal hygiene and thus can be a preventive care against various vaginal infections. This is achieved by means of undergarment with opening at crotch area and a changeable crotch piece that covers crotch opening and is replaceable as necessary. Convenient replacement with cleaner crotch piece helps in vaginal hygiene.

It includes Undergarment with an opening at the crotch area which is covered by a detachable crotch piece referred to as stripe that can be easily replaced without need for insertion or change of whole undergarment.

It includes, a front panel and rear panel with stretchable waist & thigh bands stitched together to form undergarment. Front & rear panels are stitched such that there is an open crotch area. Crotch area can be covered by detachable crotch piece (referred as Stripe) which can be fastened to the undergarment front and rear area with fasteners or adhesives

Objective of this invention is to provide convenience that Crotch opening at the above described undergarment can be covered or uncovered with a changeable crotch piece (stripe) by fastening or unfastening it to undergarment, respectively.

Another objective of the invention that Fasteners and/or adhesives used are attached on either outer or inner area of undergarment thus allowing fastening/attaching of the detachable stripe on either outer or inner panel area of the undergarment covering open crotch area of undergarment.

Further objective of invention is to allow need based, convenient replacement of Crotch piece (‘Stripe’) to maintain vaginal hygiene and act as a preventive care against any vaginal infections caused by prolonged contact with moist and unclean undergarment crotch surface.

Present invention applies to undergarment with open crotch area and convenient replacement with cleaner crotch piece easily attachable to the undergarment/panty for maintaining vaginal hygiene

Purpose of this Invention:

Vaginal discharge depends on individual body constitution. Vaginal discharge can feel wet and uncomfortable and spoil undergarment which may need replacement. Moist crotch area on undergarment can be easily changed by using fresh changeable crotch piece referred to as stripe. This stripe can be easily attached to the main undergarment with the help of adhesives or fasteners.

### BRIEF DESCRIPTION OF THE DRAWINGS

FIG. 1 is a perspective view of undergarment construction embodying my invention.

FIG. 2 is a front view of the undergarment with detachable stripe attached in its place on the undergarment with fasteners or adhesives

FIG. 3 is the rear view of the undergarment with detachable stripe attached in its place on the undergarment with fasteners or adhesives

FIG. 4 is the front view of the undergarment with open crotch area without detachable stripe as shown in FIG. 2

FIG. 5 is the rear view of the undergarment with open crotch area without detachable stripe as shown in FIG. 3

FIG. 6 is the folded open out view of the undergarment shown in FIG. 4 & FIG. 5

FIG. 7 is the inside view of the crotch stripe

FIG. 8 is the outer view of the crotch stripe

FIG. 9 illustrates the exploded perspective view of the detachable undergarment stripe

FIG. 10 is the view of the undergarment before the stripe is attached

FIG. 11 is the view after the stripe is attached to the undergarment with adhesive

### DETAILED DESCRIPTION OF THE DRAWINGS

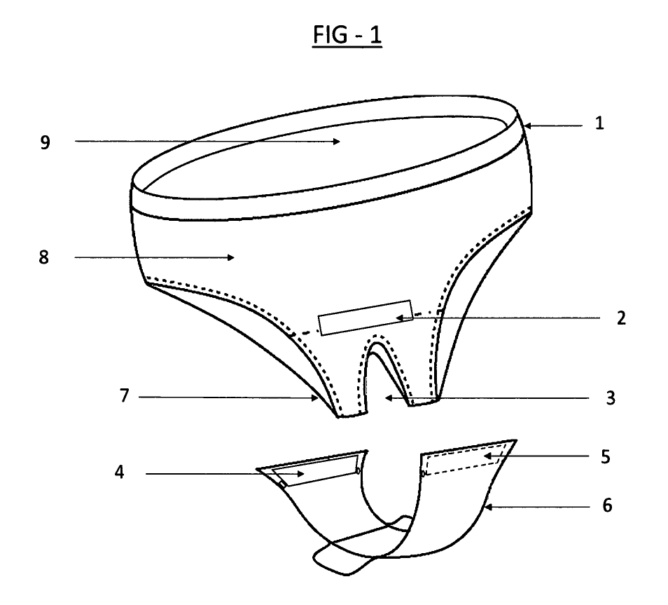

FIG. 1, is perspective view of the undergarment that embodies front panel 8 and back panel 9. It has opening at the top 10. Undergarment has elastic band 1 that connects front panel 8 and back panel 9

Elastic band 1 holds the undergarment on wearers body and worn by wearer like any conventional undergarment.

Uniqueness in this invention is the open crotch area 3. An opening 3 in the undergarment which will keep the crotch area of the wearer uncovered when worn. The size and shape of the open crotch area 3 is variable depending upon shape and size of the undergarment. This open crotch area 3 is covered with detachable stripe 6. Detachable stripe 6 can be changed by the user on need basis without changing the whole undergarment. Detachable stripe 6 can be shorter or longer in length on the basis of shape of the underwear covering open crotch area 3.

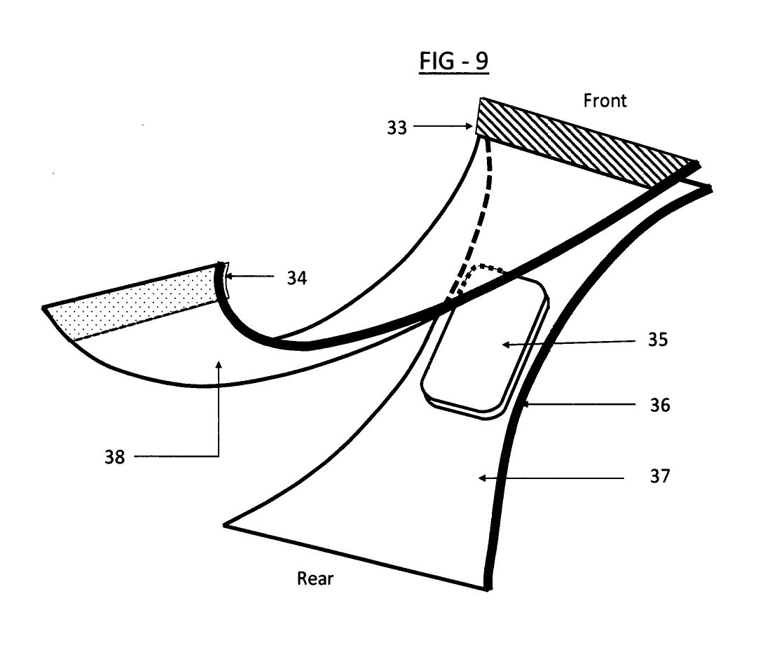

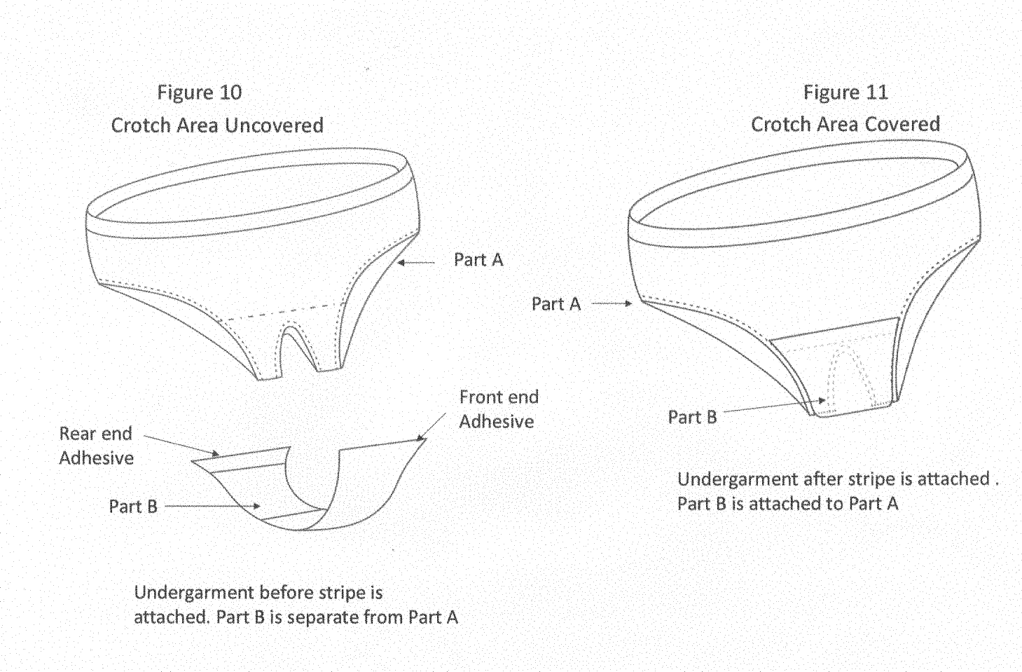

Stripe 6 is detachable and covers the open crotch area 3. Detachable stripe 6 is attached to the main undergarment with a) fasteners or b) adhesives.
a) Detachable stripe (changeable crotch piece) attached with fasteners: Fasteners like hook and loop commonly known as Velcro which typically consists of two lineal fabric strips or alternatively round dots or squares which are attached sewn or otherwise adhered to the opposing surface, particularly in this invention 2 on the front panel 8 of undergarment and 5 on front end of the detachable stripe to be fastened. One part of the fastener 2 is fastened on either exterior or interior of the front panel of undergarment and 5 on the front-end interior of the detachable stripe. Similarly, one part of the fastener is fastened on either exterior or interior of the rear panel of undergarment and 4 on the rear end of interior of detachable stripe.
b) Detachable stripe (changeable crotch piece) attached with adhesives: This drawing can be described in two parts. Part A and Part B. Refer FIG. 10. Part A is the main undergarment whereas Part B is the Changeable crotch piece or Stripe.
   Changeable crotch piece or Stripe (Part B) can be attached to main undergarment (Part A) on front and rear side with adhesives. Refer FIG. 11. Adhesive is applied on front and rear end of the stripe/Changeable Crotch piece (Part B). Refer FIG. 9, number 33 & 34.

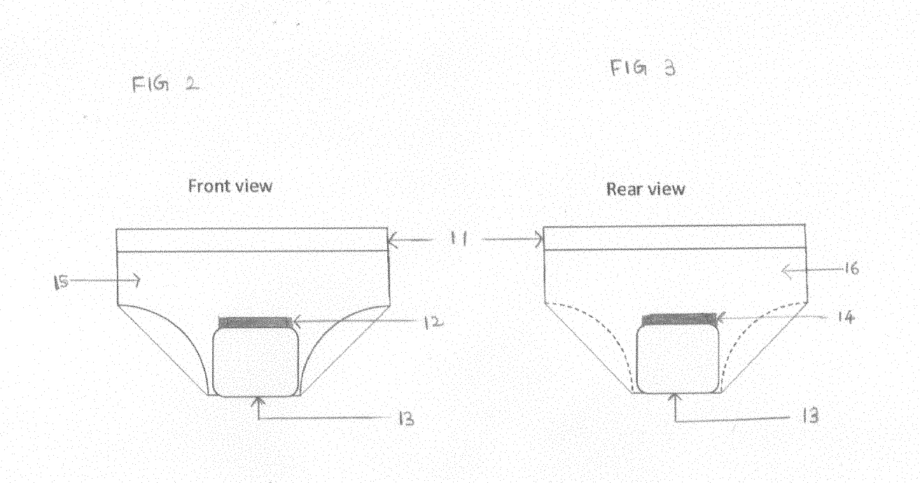

FIG. 2, is a front view of the undergarment with detachable stripe 13 attached in its place on the undergarment with fasteners or adhesives, outer view of the fastener 12 on front part of the undergarment. Elastic band 11 holds the undergarment on wearer's body. Position of the fastener 12 on front end of the undergarment can be long or short in length and is positioned on the front panel 15 of the undergarment. The position of fastener 12 on the front panel 15 is variable depending upon of shape and size of the undergarment. Purpose of this position of the fastener is to fasten the detachable stripe 13 on the front panel 15 of undergarment to cover the open crotch area 3 in FIG. 1.

FIG. 3, is the rear view of the undergarment with detachable stripe 13 attached on rear part of the undergarment with fasteners 14. Position of the fastener 14 on rear end of the undergarment can be long or short in length and positioned on rear panel of the undergarment on the basis of shape and size of the undergarment. The position of fastener 14 on the rear panel 16 is variable depending upon of shape and size of the undergarment. Purpose of this position of the fastener is to fasten the detachable stripe 13 on the rear part of the undergarment to cover the open crotch area 3 in FIG. 1.

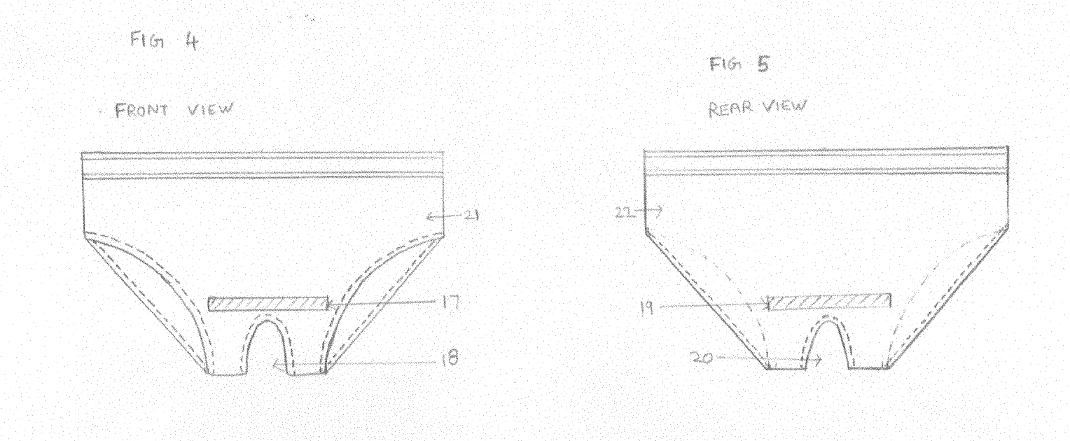

FIG. 4, is the front view of the undergarment with open crotch area 18 without detachable stripe. One component of fastener 17 is attached (sewn or otherwise adhered) to front panel 21 of the undergarment. The position of this one component of fastener 17 on the front panel 21 is variable depending upon shape and size of the undergarment. The fastener length could be long or short depending upon shape and size of the undergarment

FIG. 5, is the rear view of the undergarment with open crotch area 20 without detachable stripe. One component of fastener 19 is attached (sewn or otherwise adhered) to rear panel 22 of the undergarment. The position of this one component of fastener 19 on the rear panel 22 is variable depending upon shape and size of the undergarment. The fastener length could be long or short depending upon shape and size of the undergarment.

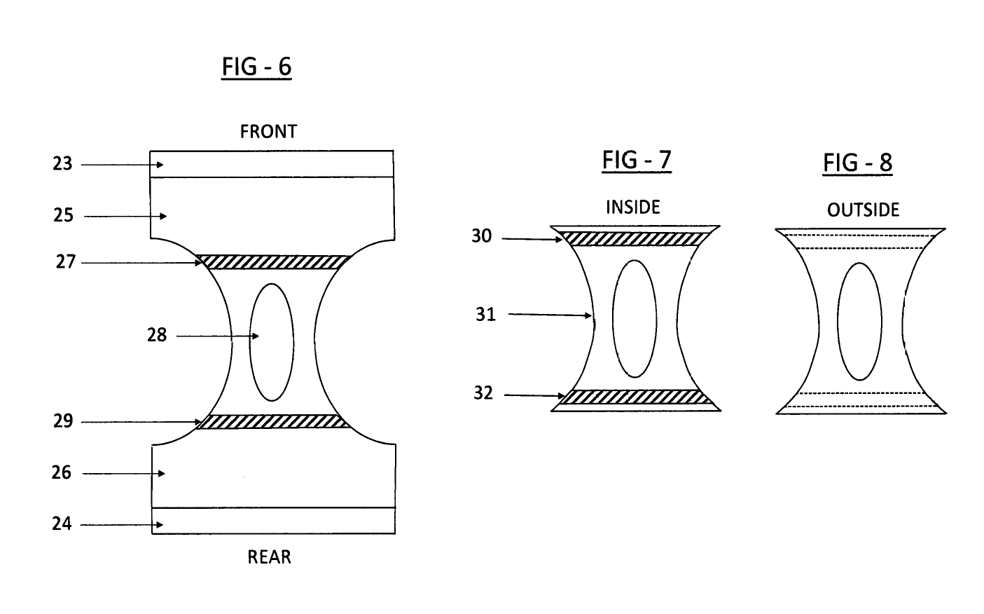

FIG. 6, is the folded open out view of the undergarment shown in FIG. 4 and FIG. 5. One half of elastic band 23 is shown attached to the front panel and another half of elastic band 24 is shown attached to rear panel 26 of the undergarment. Like any conventional undergarment front panel and rear panel are joint together by stitching. This figure also shows an opening 28 in the undergarment which will keep the crotch area of the wearer uncovered when worn. The size and shape of the open crotch area is variable depending upon shape and size of the undergarment. One component of the fastener 27 is attached to either interior or exterior side of the front panel 25 of undergarment. Similarly, one component of the fastener 29 is attached to either interior or exterior side of the rear panel 26 of undergarment.

FIG. 7, is the inside view of the detachable stripe that can be attached to the main body of the undergarment shown in FIG. 1. FIG. 7 detachable stripe 31 covers the open crotch area 28 of FIG. 6 by means of one component of the fastener 27 to the other component of the fastener 30 adhered to on front panel 25.

Similarly, detachable stripe 31 covers the open crotch area 28 of FIG. 6 by means of one component of the fastener 29 to the other component of the fastener 32 adhered to on rear panel 26.

FIG. 8, is the outer view of the detachable stripe.

FIG. 9, illustrates the exploded perspective view of the detachable undergarment stripe in FIG. 1. This detachable stripe is made of multiple layers of fabric material which will help absorb body fluids discharged from vaginal area.

The purpose of this invention of detachable stripe is to create provision for replacement of crotch area of undergarment that comes in contact with the vaginal area. Given crotch area of undergarment can soil due to various body fluid discharges a replaceable stripe will help provide cleaner surface contact with vagina helping prevent bacterial and yeast overgrowth.

FIG. 9, detachable stripe has two ends, front and rear that corresponds with front and rear end panels of the undergarment. One component of the fastener 33 on front end of the detachable stripe corresponds and is attachable to the other component of fastener on front panel of the undergarment. Similarly, one component of the fastener 34 on rear end of the detachable stripe corresponds and is attachable on the other component of fastener on rear panel of the undergarment.

In case of using adhesive as fastener, adhesive is applied on front end 33 of the stripe and rear end 34 of the stripe.

Detachable stripe consists of multiple layers of panels made from fabric, refer 37 and 38 in FIG. 9. Absorbent material 35 is placed between multiple layers of fabric to provide additional fluid absorbing capacity to detachable stripe.

1. Undergarment with opening at crotch area and an attachable, replaceable crotch piece covering crotch area for maintaining vaginal hygiene. This undergarment comprises of
a. Front panel
b. Rear panel
c. Open crotch area
d. changeable crotch piece
i. conveniently attachable to the undergarment and detachable from the undergarment from outside or
ii. conveniently attachable to the undergarment and detachable from the undergarment from inside

2. The purpose of this invention of detachable crotch piece referred to as stripe is to create provision for convenient and easy replacement of crotch area of undergarment that comes in contact with the vaginal area without replacing/changing the whole undergarment. Given crotch area of undergarment can become unhygienic due to various body fluid discharges, a replaceable crotch piece can help provide cleaner surface contact with vagina for maintaining vaginal hygiene.

3. The construction of undergarment is in two parts, Part A and Part B.
a. Part A is like conventional undergarment made from various materials for example fabric except that it will have an opening at crotch area.
i. The size and shape of the open crotch area is variable depending upon shape and size of the undergarment.
b. Part B is a crotch piece referred to as stripe attachable to the part A with any kind of fasteners or adhesives on the front and rear panel of the undergarment (Part A).
i. The size and shape of the detachable crotch piece is variable depending upon shape and size of the undergarment.
ii. The position of fastener on the front panel and rear panel is variable depending upon shape and size of the undergarment.
iii. Purpose of this position of the fastener is to fasten the detachable crotch piece on the front panel and rear panel of undergarment to cover the open crotch area

4. Crotch piece (Part B) could be fastened to undergarment (Part A) with various types of fasteners such as hook and loop fasteners, adhesives, hook and eye, snap fasteners, buttons, threads, zips, buckles or any other type of fasteners which can hold crotch piece (Part B) to undergarment (Part A).

5. The position of fastener on the front panel and rear panel of undergarment (Part A) is variable depending upon shape and size of the undergarment (Part A) and crotch piece (Part B). Purpose of this position of the fastener is to fasten the detachable crotch piece (Part B) on the front panel and rear panel of undergarment (Part A) to cover the open crotch area.

6. Crotch piece (Part B) is made from multiple layers of fabric material which can absorb small quantity of body fluids discharged from vaginal area.
